SEQUENCE LISTING (1 sequence)

SEQ ID NO: 1 - protein, 136 aa, Escherichia coli
  REGION 1..136: note = 1,4-dihydroxy-2-naphthoyl-CoA hydrolase (Ydil); UniProt: P77781
  source 1..136: note = subspecies K12
MIWKRKITLEALNAMGEGNMVGFLDIRFEHIGDDTLEATMPVDSRTKQPFGLLHGGASVVLAESIGSVAGYLCTEGEQKVVGLEINANHVRSAREGRVRGVCKPLHLGSRHQVWQIEIFDEKGRLCCSSRLTTAIL

(19) United States
(12) Patent Application Publication
(10) Pub. No.: US 2025/0333769 A1
(43) Pub. Date: Oct. 30, 2025

(54) ACYL-COA HYDROLASE VARIANTS

(71) Applicant: Global Bioenergies, Evry Cedex (FR)

(72) Inventors: Benoît Villiers, Tours (FR); François Stricher, Wolfisheim (FR)

(73) Assignee: Global Bioenergies, Evry Cedex (FR)

(21) Appl. No.: 18/719,199

(22) PCT Filed: Dec. 12, 2022
(86) PCT No.: PCT/EP2022/085392

§ 371 Date: Jun. 12, 2024

(30) Foreign Application Priority Data
Dec. 13, 2021 (EP) 21213999.2

(51) Int. Cl. C12P 7/40; C12N 9/16

(52) U.S. Cl. CPC C12P 7/40; C12N 9/16; C12Y 301/02001

(57) ABSTRACT

Described are acyl-CoA hydrolase (ACH) variants variants showing an improved activity in converting 3-methylcrotonyl-CoA into 3-methylcrotonic acid or an increased activity in converting crotonyl-CoA into crotonic acid as well as methods for the production of 3-methylcrotonic acid or isobutene or crotonic acid using such enzyme variants.

Described are acyl-CoA hydrolase (ACH) variants showing an improved activity in converting 3-methylcrotonyl-CoA into 3-methylcrotonic acid or an increased activity in converting crotonyl-CoA into crotonic acid (also called butenoic acid), as well as methods for the production of 3-methylcrotonic acid or crotonic acid or isobutene using such enzyme variants.

A large number of chemical compounds are currently derived from petrochemicals. Alkenes (such as ethylene, propylene, the different butenes, or else the pentenes, for example) are used in the plastics industry, for example for producing polypropylene or polyethylene, and in other areas of the chemical industry and that of fuels.

Butylene exists in four forms, one of which, isobutene (also referred to as isobutylene), enters into the composition of methyl-tert-butyl-ether (MTBE), an anti-knock additive for automobile fuel. Isobutene can also be used to produce isooctene, which in turn can be reduced to isooctane (2,2,4-trimethylpentane); the very high octane rating of isooctane makes it the best fuel for so-called “gasoline” engines. Alkenes such as isobutene are currently produced by catalytic cracking of petroleum products (or by a derivative of the Fischer-Tropsch process in the case of hexene, from coal or gas). The production costs are therefore tightly linked to the price of oil. Moreover, catalytic cracking is sometimes associated with considerable technical difficulties which increase process complexity and production costs.

The production by a biological pathway of alkenes such as isobutene is called for in the context of a sustainable industrial operation in harmony with geochemical cycles. The first generation of biofuels consisted in the fermentative production of ethanol, as fermentation and distillation processes already existed in the food processing industry. The production of second-generation biofuels is in an exploratory phase, encompassing in particular the production of long chain alcohols (butanol and pentanol), terpenes, linear alkanes and fatty acids. Two recent reviews provide a general overview of research in this field: Ladygina et al. (Process Biochemistry 41 (2006), 1001) and Wackett (Current Opinions in Chemical Biology 21 (2008), 187).

Different routes for the enzymatic generation of isobutene have previously been described; see, e.g., Fujii et al. (Appl. Environ. Microbiol. 54 (1988), 583); Gogerty et al. (Appl. Environm. Microbiol. 76 (2010), 8004-8010) and van Leeuwen et al. (Appl. Microbiol. Biotechnol. 93 (2012), 1377-1387) and WO2010/001078. In addition to these routes, there are also alternative routes for the provision of isobutene utilizing the enzymatic conversion of 3-methylcrotonic acid into isobutene by a decarboxylation reaction. WO2017/191239 and WO2020/007886 describe enzyme variants based on FDC enzymes of Hypocrea atroviridis and Streptomyces sp. 769 which show an improved ability to convert 3-methylcrotonic acid into isobutene.

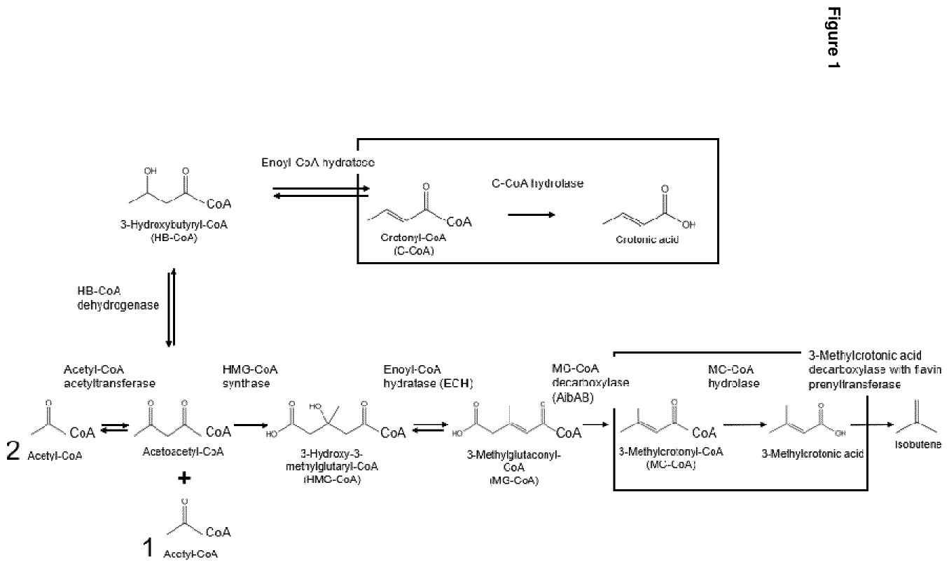

WO2017/085167 describes routes for enzymatically producing the precursor of isobutene, i.e. 3-methylcrotonic acid, from acetyl-CoA. According to one of the described routes two molecules of acetyl-CoA are first converted into acetoacetyl-CoA, which is further converted into 3-hydroxy-3-methylglutaryl-CoA, which is then further converted into 3-methylglutaconyl-CoA, which is converted into 3-methylcrotonyl-CoA, which is finally converted into 3-methylcrotonic acid (see FIG. 1).

α,β-unsaturated carboxylic acids (α,β-UCAs) are widely used in organic synthesis both as intermediates and final products. Owing to their application in food, polymer, perfume and medicine industry, they are synthesized on a commercial scale.

Crotonic acid (butenoic acid), in particular, is an important C4 α,β-UCA and has wide industrial applications including pharmaceuticals (Zeiller J -J, Dumas H, Guyard-Dangremont V, Berard I, Contard F, Guerrier D, Ferrand G, Bonhomme Y (2012) Butenoic acid derivatives, processes for the preparation thereof, pharmaceutical compositions comprising them, and use for the treatment of dyslipidaemia, atherosclerosis and diabetes. U.S. Pat. No. 8,247,448), agrochemicals (Saraydin, D., Karadağ, E. & Güven, O. The releases of agrochemicals from radiation induced acrylamide/crotonic acid hydrogels. Polymer Bulletin 41, 577-584 (1998). https://doi.org/10.1007/s002890050404), cosmetics (Rollat I, Samain H, Morel O (2006) Reshapable hair styling composition comprising (meth)acrylic copolymers of four or more monomers. U.S. Pat. No. 7,122,175), and resins (Wakaki S, Yamamoto T, Enoki H (2008) Stabilizing agent for chlorine containing polymer used for chlorine containing polymer composition, contains epoxy-group containing acrylic resin, amino crotonic acid ester, polyhydric alcohol and/or hindered amine or phenyl indole. WO2008087784-A1; JP2008195912-A; JP5192182-B2).

To replace fossil-based production of crotonic acid and obtain a more sustainable process, crotonic acid biosynthesis has been developed in different organisms including Escherichia coli (Dellomonaco, C., Clomburg, J., Miller, E. et al. Engineered reversal of the β-oxidation cycle for the synthesis of fuels and chemicals. Nature 476, 355-359 (2011). https://dol.org/10.1038/nature10333) (Liu X, Yu H, Jiang X, Ai G, Yu B, Zhu K. Biosynthesis of butenoic acid through fatty acid biosynthesis pathway in Escherichia coli. Appl Microbiol Biotechnol. 2015 February;99(4):1795-804. doi: 10.1007/s00253-014-6233-2.) (Kim S, Cheong S, Gonzalez R. Engineering Escherichia coli for the synthesis of short-and medium-chain α,β-unsaturated carboxylic acids. Metab Eng. 2016 July;36:90-98. doi: 10.1016/j.ymben.2016.03.005) (Ji X, Zhao H, Zhu H, Zhu K, Tang S Y, Lou C. CRISPRi/dCpf1-mediated dynamic metabolic switch to enhance butenoic acid production in Escherichia coli. Appl Microbiol Biotechnol. 2020 June;104(12):5385-5393. doi: 10.1007/s00253-020-10610-2.), Yarrowia lipolytica (Wang L, Zong Z, Liu Y, Zheng M, Li D, Wang C, Zheng F, Madzak C, Liu Z. Metabolic engineering of Yarrowia lipolytica for the biosynthesis of crotonic acid. Bioresour Technol. 2019 September;287:121484. doi: 10.1016/j.biortech.2019.121484.), Methylomicrobium buryatense 5GB1C (Garg S, Wu H, Clomburg J M, Bennett G N. Bioconversion of methane to C-4 carboxylic acids using carbon flux through acetyl-CoA in engineered Methylomicrobium buryatense 5GB1C. Metab Eng. 2018 July;48:175-183. doi: 10.1016/j.ymben.2018.06.001), Methylobacterium extorquens AM1 (Schada von Borzyskowski L, Sonntag F, Pöschel L, Vorholt J A, Schrader J, Erb T J, Buchhaupt M. Replacing the Ethylmalonyl-CoA Pathway with the Glyoxylate Shunt Provides Metabolic Flexibility in the Central Carbon Metabolism of Methylobacterium extorquens AM1. ACS Synth Biol. 2018 Jan. 19;7(1):86-97. doi: 10.1021/acssynbio.7b00229).

For crotonic acid biosynthesis, an engineered reversal of the β-oxidation (r-BOX) cycle in Escherichia coli using Ydil (from Escherichia coli) as the cycle-terminating enzyme seems one of the most promising approach. But despite being the most efficient and specific thioesterase from Escherichia coli on crotonyl-CoA, Ydil's activity on this substrate will need further improvement to allow an industrial application, especially considering crotonyl-CoA hydrolysis is the only irreversible step in the pathway between acetyl-CoA and crotonic acid (Kim S, Cheong S, Gonzalez R. Engineering Escherichia coli or the synthesis of short- and medium-chain α,β-unsaturated carboxylic acids. Metab Eng. 2016 July; 36:90-98. doi: 10.1016/j.ymben.2016.03.005).

Although the above means and methods allow to produce the precursor 3-methylcrotonic acid (which is enzymatically further converted into isobutene) or crotonic acid from acetyl-CoA, there is still a need for improvements, in particular as regards a further increase in efficiency of the respective processes so as to make them more suitable for industrial purposes.

The present application addresses this need by providing the embodiments as defined in the claims.

In particular, the present invention addresses this need by providing improved enzymes which catalyse the conversion of 3-methylcrotonyl-CoA into 3-methylcrotonic acid, i.e. one of the reactions in the above described pathway leading from acetyl-CoA to 3-methylcrotonic acid. By providing such improved enzyme variants, the production of 3-methylcrotonic acid can be improved and rendered more efficient, which, in turn, allows to improve the production of isobutene from 3-methylcrotonic acid.

It could be shown that such improved enzymes also show an increased activity of converting crotonyl-CoA into crotonic acid thereby allowing an efficient production of crotonic acid.

Thus, the present invention provides a variant of an acyl-CoA hydrolase (ACH) showing an improved activity in converting 3-methylcrotonyl-CoA into 3-methylcrotonic acid over the corresponding ACH from which it is derived as defined in the claims.

An improved enzyme variant or an enzyme variant capable of catalyzing a reaction with increased activity is defined as an enzyme variant which differs from the wildtype enzyme and which catalyzes the conversion of 3-methylcrotonyl-CoA into 3-methylcrotonic acid so that the specific activity of the enzyme variant is higher than the specific activity of the wildtype enzyme for at least one given concentration of 3-methylcrotonyl-CoA (preferably any 3-methylcrotonyl-CoA concentration higher than 0 M and up to 1 M). A specific activity is defined as the number of moles of substrate converted to moles of product by unit of time by mole of enzyme. K_{cat} (turnover number) is the specific activity at saturating concentration of substrate.

In the context of the present invention, an “improved activity” means that the activity of the enzyme in question is at least 10%, preferably at least 20%, more preferably at least 30% or 50%, even more preferably at least 70% or 80% and particularly preferred at least 90% or 100% higher than that of the enzyme from which the variant is derived, preferably higher than that of the enzyme represented by SEQ ID NO:1. In even more preferred embodiments the improved activity may be at least 150%, at least 200%, at least 300%, at least 750% or at least 1000% higher than that of the corresponding enzyme from which the variant is derived, preferably higher than that of the enzyme represented by SEQ ID NO:1. In a particularly preferred embodiment, the activity is measured by using an assay with purified enzyme and chemically synthesized substrates, as described below. The improved activity of a variant can be measured as a higher production of 3-methylcrotonic acid in a given time under defined conditions, compared with the parent enzyme. This improved activity can result from a higher turnover number, e.g. a higher kcat value. It can also result from a lower Km value. It can also result from a higher kcat/Km value. Finally, it can result from a higher solubility, or stability of the enzyme. The degree of improvement can be measured as the improvement in production of 3-methylcrotonic acid. The degree of improvement can also be measured in terms of kcat improvement, of kcat/Km improvement, or in terms of Km decrease, in terms of soluble protein production or in terms of protein stability.

In another embodiment, the enzyme variants which the present invention provides are capable of converting 3-methylcrotonyl-CoA into 3-methylcrotonic acid with an activity which is at least 1.10 times as high compared to the turnover rate of the corresponding wild type enzyme having the amino acid sequence as shown in SEQ ID NO:1. In a more preferred embodiment, the enzyme variants which are capable of converting 3-methylcrotonyl-CoA into 3-methylcrotonic acid have a turnover rate (i.e., a k_{cat}-value) which is at least 2 times, at least 3 times, at least 5 times or even at least 10 times as high compared to the turnover rate of the corresponding wild type enzyme having the amino acid sequence as shown in SEQ ID NO:1. In even more preferred embodiments, the turnover rate is at least 100 times or even at least 500 times as high compared to that of the corresponding wild type enzyme having the amino acid sequence as shown in SEQ ID NO:1.

Such enzyme variants are obtained by effecting mutations at specific positions in the amino acid sequence of an acyl-CoA hydrolase (ACH) and the variants obtained by effecting such mutations show an improved activity in catalyzing the conversion of 3-methylcrotonyl-CoA into 3-methylcrotonic acid. The activity of an enzyme capable of converting 3-methylcrotonyl-CoA into 3-methylcrotonic acid may be determined by methods known to the person skilled in the art. In one embodiment, this activity is determined as described in the Examples appended hereto. In a particular embodiment this activity can be measured by incubating the enzyme, preferably a cell lysate containing the overexpressed recombinant protein, in vitro. Alternatively, a (partly) purified enzyme can be used or an in vivo assay.

More specifically, the activity of the ACH variants for the conversion of 3-methylcrotonyl-CoA 3-methylcrotonic acid can be assessed by an enzymatic in vitro assay based on purified proteins and on the detection of 3-methylcrotonic acid, e.g. by using HPLC. An example of a corresponding assay is, e.g., described in WO2017/085167, Example 5.

As described above, it could be shown that such improved enzyme variants which show an increased activity of converting 3-methylcrotonyl-CoA into 3-methylcrotonic acid also show an increased activity of converting crotonyl-CoA into crotonic acid thereby allowing an efficient production of crotonic acid. As regards the definition of an improved or increased activity, the same applies for the reaction of the conversion of crotonyl-CoA into crotonic acid as has been set forth above for the conversion of 3-methylcrotonyl-CoA into 3-methylcrotonic acid.

The ACH variant to be tested can, e.g., be provided according to the following protocol for an in vitro screening approach as described in the appended Examples.

The polynucleotide sequences coding for the different mutants identified during the evolution of ACH (in vivo screening) are cloned alone in a pRSFDuet (Novagen) expression vector with a polynucleotide tag in 5′ coding for a 6His purification tag. Purification and characterization of ACH mutants with a direct in vitro assay can, e.g., be carried out as follows:

ACH mutants are purified by affinity chromatography (Ni-NTA) from bacterial cultures of BL21 (DE3) cells containing the gene cloned into a pRSFDuet (Novagen) expression vector with a polynucleotide tag in 5′ coding for a 6-His purification tag. The purified ACH mutants are tested at 1 μg/ml or 0.1 μg/ml in a direct in vitro assay with a buffered solution containing 50 mM Tris-HCl pH7.5, 20 mM NaCl, 100 mM KCl, 2 mM MgCl2, 1 mM 3-methylcrotonyl-CoA (MC-CoA) (Endotherm) (to measure 3-methylcrotonyl-CoA (MC-CoA) hydrolase activity) or 1 mM Crotonyl-CoA (Sigma-Aldrich) (to measure Crotonyl-CoA hydrolase activity). The reactions are incubated 30 min at 36° C. in a water bath, stopped with acetonitrile and the produced 3-Methylcrotonic acid (Sigma-Aldrich) (to measure MC-CoA hydrolase activity) or Crotonic acid (TCI chemicals) (to measure Crotonyl-CoA hydrolase activity) are quantified by HPLC with a calibration curve. The samples are injected on a Zorbax SB-Aq column (Agilent) at 30° C. and eluted with 8.4 mM H2SO4 and a gradient of acetonitrile.

By providing the above described enzyme variant, the present invention allows to dramatically increase the production efficiency of 3-methylcrotonic acid from 3-methylcrotonyl-CoA or the production efficiency of crotonic acid from crotonyl-CoA.

The term “acyl-CoA hydrolase (ACH)” refers to an enzyme which is classified as EC 3.1.2.20. Acyl-CoA hydrolases are enzymes which catalyze the following reaction:

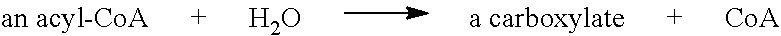

This activity can be measured by methods known in the art and as described above.

This enzyme occurs in a variety of organism, including eukaryotic and prokaryotic organisms, such as plants, animals, fungi and bacteria. The enzyme has, e.g., been described in Arabidopsis thaliana, Rhodotorula aurantiaca, Bumilleriopsis filiformis, Eremosphaera viridis, Euglena gracilis, Mus musculus, Rattus norvegicus, Homo sapiens, Sus, scrofa, Bos taurus, Cais lupus familiaris, Cavia porcellus, Cricetus griseus, Drosophila melanogaster, Anas platyrhynchos, Gallus gallus, Caenorhabditis elegans, Saccharomyces cerevisia, Candida rugosa, Escherichia coli, Haemophilus influenzae, Xanthomonas campestris, Streptomyces sp., Streptomyces coelicolor, Alcaligenes faecalis, Pseudomonas aeruginosa, Pseudomonas putida, Amycolatopsis mediterranei, Acinetobacter calcoaceticus, Helicobacter pylori, Rhodobacter spaeroides and Mycobacterium phlei. Specific examples are the acyl-CoA hydrolases from Escherichia coli, from Pseudomonas putida or from Haemophilus influenza, e.g. the YciA enzyme from E. coli or its closely related homolog HI0827 from Haemophilus influenza (Zhuang et al., Biochemistry 47 (2008), 2789-2796). The YciA enzyme from Haemophilus influenza is described to catalyze the hydrolysis of propionyl-CoA into propionic acid (Zhuang et al., Biochemistry 47 (2008), 2789-2796). Another example is the enzyme from Homo sapiens (UniProt: Q9NPJ3) which is described to hydrolyze propionyl-CoA (Cao et al., Biochemistry 48 (2009), 1293-1304). Other examples are the enzymes acyl-CoA thioester hydrolase from E. coli (Uniprot POA8Z0), acyl-CoA thioesterase 2 from E. coli (Uniprot POAGG2), acyl-CoA thioesterase II from Pseudomonas putida (Uniprot Q88DR1).

The acyl-CoA hydrolase (ACH) variant can be derived from any of the ACHs mentioned above.

In a preferred embodiment, the acyl-CoA hydrolase variant derived from an enzyme belonging to the family of 1,4-dihydroxy-2-naphthoyl-CoA hydrolases. Enzymes of this family of 1,4-dihydroxy-2-naphthoyl-CoA hydrolases are known to catalyze the following reaction:

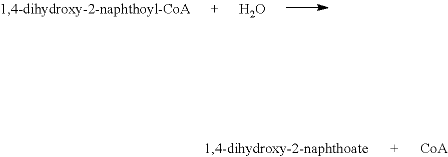

These enzymes are also often referred to as Ydil thioesterases. Enzymes of this family occur in a variety of organisms and have, e.g., been described in Escherichia coli and Salmonella enterica. Thus, in a particularly preferred embodiment the ACH is derived from Escherichia coli or from Salmonella enterica, more preferably the acyl-CoA hydrolase (ACH) variant is derived from the ydil enzyme of Escherichia coli (Uniprot P77781; SEQ ID NO:1).

It had previously been described that acyl-CoA hydrolase (ACH) enzymes, in particular enzymes belonging to the family of 1,4-dihydroxy-2-naphthoyl-CoA hydrolases can be used to convert 3-methylcrotonyl-CoA into 3-methylcrotonic acid (see, e.g., WO2017/085167).

The present invention provides now improved variants of acyl-CoA hydrolases (ACH) which are capable of converting 3-methylcrotonyl-CoA into 3-methylcrotonic acid or which show an increased activity of converting crotonyl-CoA into crotonic acid. The inventors used as a model enzyme the ACH “Ydli (menl)” from Escherichia coli (Uniprot P77781) shown in SEQ ID NO: 1 and could show that it is possible to provide variants of this enzyme which show an increased activity with respect to the conversion of 3-methylcrotonyl-CoA into 3-methylcrotonic acid. The identified variants also showed an increased activity of converting crotonyl-CoA into crotonic acid.

The model enzyme, i.e., the ACH of Escherichia coli, as used by the inventors has the amino acid sequence as shown in SEQ ID NO:1.

In one preferred embodiment the variants of the present invention are characterized by the feature that they are derived from an ACH, more preferably from an ACH having the amino acid sequence shown in SEQ ID NO:1 or a highly related sequence (at least 60% identical) and in which mutations are effected at one or more of the positions indicated further below and by the feature that they show the ability to convert 3-methylcrotonyl-CoA into 3-methylcrotonic acid and that they can do this with an improved activity. In a preferred embodiment the variant according to the present invention is derived from a sequence which shows at least 65%, more preferably at least 70%, 75%, 80% or 85%, more preferably at least 87%, even more preferably at least 90%, 91%, 92%, 93%, 94% or at least 95% sequence identity to SEQ ID NO:1 and in which one or more substitutions and/or deletions and/or insertions at the positions indicated herein have been effected.

However, the teaching of the present invention is not restricted to the ACH enzyme of Escherichia coli shown in SEQ ID NO: 1 which had been used as a model enzyme but can be extended to ACH enzymes from other organisms or to enzymes which are structurally related to SEQ ID NO:1 such as, e.g., truncated variants of the enzyme. Thus, the present invention also relates to variants of ACH which are structurally related to the Escherichia coli sequence (SEQ ID NO: 1) and which show one or more substitutions and/or deletions and/or insertions at positions corresponding to any of the positions as indicated herein below. The term “structurally related” refers to ACH which show a sequence identity of at least n % to the sequence shown in SEQ ID NO: 1 with n being an integer between 60 and 100, preferably 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99. In a preferred embodiment the structurally related ACH stems from a bacterium, more preferably from a gram-negative bacterium, even more preferably of the genus Escherichia, Salmonella or Haemophilus.

Thus, in one embodiment, the variant of an ACH according to the present invention has (or preferably is derived from) a sequence which is at least n % identical to SEQ ID NO: 1 with n being an integer between 60 and 100, preferably 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99, and it has (a) substitution(s) and/or (a) deletion(s) and/or (an) insertion(s) at a position as indicated herein. When the sequences which are compared do not have the same length, the degree of identity either refers to the percentage of amino acid residues in the shorter sequence which are identical to amino acid residues in the longer sequence or to the percentage of amino acid residues in the longer sequence which are identical to amino acid residues in the shorter sequence. Preferably, it refers to the percentage of amino acid residues in the shorter sequence which are identical to amino acid residues in the longer sequence. The degree of sequence identity can be determined according to methods well known in the art using preferably suitable computer algorithms such as CLUSTAL. When using the Clustal analysis method to determine whether a particular sequence is, for instance, at least 60% identical to a reference sequence default settings may be used.

In a preferred embodiment Clustal Omega (Madeira F, Park Y M, Lee J, et al. The EMBL-EBI search and sequence analysis tools APIs in 2019. Nucleic Acids Research. 2019 July;47 (W1): W636-W641. DOI: 10.1093/nar/gkz268. PMID: 30976793; PMCID: PMC6602479) is used for the comparison of amino acid sequences. In the case of pairwise comparisons/alignments, the following default settings are preferably chosen: Program: clustalo; Version: 1.2.4; Input Parameters: Output guide tree: true; Output distance matrix: false; Dealign input sequences: false; mBed-like clustering guide tree: true; mBed-like clustering iteration: true; Number of iterations: 0; Maximum guide tree iterations: −1; Maximum HMM iterations: −1; Output alignment format: clustal_num; Output order: aligned; Sequence Type: protein. Preferably, the degree of identity is calculated over the complete length of the sequence.

Amino acid residues located at a position corresponding to a position as indicated herein in the amino acid sequence shown in SEQ ID NO:1 can be identified by the skilled person by methods known in the art. For example, such amino acid residues can be identified by aligning the sequence in question with the sequence shown in SEQ ID NO:1 and by identifying the positions which correspond to the above or below indicated positions of SEQ ID NO:1. The alignment can be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences.

In a preferred embodiment Clustal Omega (Madeira F, Park Y M, Lee J, et al. The EMBL-EBI search and sequence analysis tools APIs in 2019. Nucleic Acids Research. 2019 July;47 (W1): W636-W641. DOI: 10.1093/nar/gkz268. PMID: 30976793; PMCID: PMC6602479) is used for the comparison of amino acid sequences. In the case of pairwise comparisons/alignments, the following default settings are preferably chosen: Program: clustalo; Version: 1.2.4; Input Parameters: Output guide tree: true; Output distance matrix: false; Dealign input sequences: false; mBed-like clustering guide tree: true; mBed-like clustering iteration: true; Number of iterations: 0; Maximum guide tree iterations: −1; Maximum HMM iterations: −1; Output alignment format: clustal_num; Output order: aligned; Sequence Type: protein. When the amino acid sequences of ACHs are aligned by means of such a method, regardless of insertions or deletions that occur in the amino acid sequences, the positions of the corresponding amino acid residues can be determined in each of the ACHs.

In the context of the present invention, “substituted with another amino acid residue” means that the respective amino acid residues at the indicated position can be substituted with any other possible amino acid residues, e.g. naturally occurring amino acids or non-naturally occurring amino acids (Brustad and Arnold, Curr. Opin. Chem. Biol. 15 (2011), 201-210), preferably with an amino acid residue selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Preferred substitutions for certain positions are indicated further below. Moreover, the term “substituted” or “substitution” also means that the respective amino acid residue at the indicated position is modified.

Such modifications include naturally occurring modifications and non-naturally occurring modifications. Naturally occurring modifications include but are not limited to eukaryotic post-translational modification, such as attachment of functional groups (e.g. acetate, phosphate, hydroxyl, lipids (myristoylation of glycine residues) and carbohydrates (e.g. glycosylation of arginine, asparagine etc.). Naturally occurring modifications also encompass the change in the chemical structure by citrullination, carbamylation and disulphide bond formation between cysteine residues; attachment of co-factors (FMN or FAD that can be covalently attached) or the attachement of peptides (e.g. ubiquitination or sumoylation).

Non-naturally occurring modifications include, e.g., in vitro modifications such as biotinylation of lysine residue or the inclusion of non-canonical amino acids (see Liu and Schultz, Annu. Rev. Biochem. 79 (2010), 413-44 and Wang et al., Chem. Bio. 2009 Mar. 27; 16 (3), 323-336; doi: 101016/jchembiol.2009.03.001).

In the context of the present invention, “deleted” or “deletion” means that the amino acid at the corresponding position is deleted.

In the context of the present invention, “inserted” or “insertion” means that at the respective position one or two, preferably one amino acid residue is inserted after the indicated position.

The present invention provides a variant of an acyl-CoA hydrolase (ACH) showing an improved activity in converting 3-methylcrotonyl-CoA into 3-methylcrotonic acid over the corresponding ACH from which it is derived or showing an improved activity in converting crotonyl-CoA into crotonic acid over the corresponding ACH from which it is derived, wherein the ACH variant is characterized in that it shows one or more substitutions, deletions and/or insertions in comparison to the corresponding sequence from which it is derived and wherein these substitutions, deletions and/or insertions occur at one or more of the positions corresponding to positions 68, 131 and 21 in the amino acid sequence shown in SEQ ID NO:1. In a preferred embodiment such a variant also shows an improved activity in both above-mentioned reactions.

The present invention relates in a preferred embodiment to an ACH variant having an amino acid sequence as shown in SEQ ID NO:1 or an amino acid sequence having at least 60% sequence identity to SEQ ID NO:1, in which one or more amino acid residues at a position selected from the group consisting of positions 68, 131 and 21 in the amino acid sequence shown in SEQ ID NO:1 or at a position corresponding to any of these positions, are substituted with another amino acid residue or deleted or wherein an insertion has been effected at one or more of these positions and wherein said ACH variant has an improved activity in converting 3-methylcrotonyl-CoA into 3-methylcrotonic acid or an improved activity in converting crotonyl-CoA into crotonic acid. In a preferred embodiment such a variant also shows an improved activity in both above-mentioned reactions.

According to one embodiment, the present invention relates to any of the above-described ACH variants having an amino acid sequence as shown in SEQ ID NO:1 or an amino acid sequence having at least 60% sequence identity to SEQ ID NO:1 in which
(1) an amino acid residue at position 68 in the amino acid sequence shown in SEQ ID NO: 1 or at a position corresponding to this position, is deleted or substituted with leucine, isoleucine, methionine or phenylalanine; and/or
(2) an amino acid residue at position 131 in the amino acid sequence shown in SEQ ID NO: 1 or at a position corresponding to this position, is deleted or substituted with histidine; and/or
(3) an amino acid residue at position 21 in the amino acid sequence shown in SEQ ID NO: 1 or at a position corresponding to this position, is deleted or substituted with isoleucine.

The invention also relates to variants as defined in (1) to (3) hereinabove, wherein the amino acid residue indicated as substituting the amino acid residue at the position in SEQ ID NO: 1 or as being inserted at a certain position is not that particular amino acid residue but an amino acid residue which is conservative in relation to the indicated substituting amino acid.

Whether an amino acid is conservative with respect to another amino acid can be judged according to means and methods known in the art and as described herein above. One possibility is the PAM 250 matrix; alternatively, the Blosum Family Matrices can be used.

In a preferred embodiment the present invention relates in particular to a variant of an acyl-CoA hydrolase (ACH) showing an improved activity in converting 3-methylcrotonyl-CoA into 3-methylcrotonic acid or an improved activity in converting crotonyl-CoA into crotonic acid over the acyl-CoA hydrolase (ACH) of SEQ ID NO: 1, wherein the acyl-CoA hydrolase (ACH) is characterized in that:
(a) it has a sequence identity of at least 60% to the sequence shown in SEQ ID NO: 1; and
(b) it shows one or more substitutions at a position selected from the group consisting of positions 68, 131 and 21 in the amino acid sequence shown in SEQ ID NO:1 or at a position corresponding to any of these positions.

In a preferred embodiment such a variant also shows an improved activity in both above-mentioned reactions.

As regards the preferred modifications at the different positions, in particular in connection with substitutions and insertions, the same applies as has been set forth above.

In one preferred embodiment, the ACH variant according to the invention showing an improved activity in converting 3-methylcrotonyl-CoA into 3-methylcrotonic acid (or an improved activity in converting crotonyl-CoA into crotonic acid) is characterized in that it contains at least one deletion, substitution and/or insertion wherein the deletion/insertion/substitution is at position 68 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 131 and 21 in the amino acid sequence shown in SEQ ID NO:1.

As regards the preferred embodiments of substitutions, deletions and/or insertions at a particular position, the same applies as has been set forth herein above.

In a further preferred embodiment, the ACH variant according to the invention showing an improved activity in converting 3-methylcrotonyl-CoA into 3-methylcrotonic acid (or an improved activity in converting crotonyl-CoA into crotonic acid) is characterized in that it contains at least one deletion, substitution and/or insertion wherein the deletion/insertion/substitution is at position 131 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 68, and 21 in the amino acid sequence shown in SEQ ID NO:1.

As regards the preferred embodiments of substitutions, deletions and/or insertions at a particular position, the same applies as has been set forth herein above.

In a further preferred embodiment, the ACH variant according to the invention showing an improved activity in converting 3-methylcrotonyl-CoA into 3-methylcrotonic acid (or an improved activity in converting crotonyl-CoA into crotonic acid) is characterized in that it contains at least one deletion, substitution and/or insertion wherein the deletion/insertion/substitution is at position 21 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 68, and 131 in the amino acid sequence shown in SEQ ID NO:1.

As regards the preferred embodiments of substitutions, deletions and/or insertions at a particular position, the same applies as has been set forth herein above.

In a more preferred embodiment, the ACH variant according to the invention showing an improved activity in converting 3-methylcrotonyl-CoA into 3-methylcrotonic acid (or an improved activity in converting crotonyl-CoA into crotonic acid) is characterized in that contains at least two deletions, substitutions and/or insertions wherein one deletion/insertion/substitution is at position 68 in the amino acid sequence shown in SEQ ID NO:1 or at a position corresponding to this position and another deletion/insertion/substitution is at position 131 in the amino acid sequence shown in SEQ ID NO:1 or at a position corresponding to this position. Preferably, such a variant further has one or more substitutions, deletions and/or insertions in comparison to the corresponding sequence from which it is derived and wherein these substitutions, deletions and/or insertions occur at one or more of the positions corresponding to positions 21 in the amino acid sequence shown in SEQ ID NO:1. It is particularly preferred that such a variant which contains modifications at positions 68 and 131 in the amino acid sequence shown in SEQ ID NO:1 or at positions corresponding to these positions furthermore shows a substitution, deletion or substitution at position 21 in the amino acid sequence shown in SEQ ID NO:1 or at a position corresponding to this position.

In preferred embodiments, the ACH variant according to the invention showing an improved activity in converting 3-methylcrotonyl-CoA into 3-methylcrotonic acid (or an improved activity in converting crotonyl-CoA into crotonic acid) is characterized in that it contains two or more modifications. Preferred combinations of positions at which such modifications occur are:
68 and 131
68 and 21
131 and 21
68 and 131 and 21

In more preferred embodiments, an ACH variant which contains two or more modifications at positions as indicated above, contains the following modifications:
68→leucine
131→histidine
21→isoleucine

The present invention also relates to a method for providing a variant of an ACH wherein said variant shows an improved activity of converting 3-methylcrotonyl-CoA into 3-methylcrotonic acid (or an improved activity in converting crotonyl-CoA into crotonic acid), said method comprising the step of effecting one or more changes in the sequence of the ACH wherein said change(s) is/are effected at one or more amino acid positions selected from the group consisting of the amino acid positions corresponding to positions 68, 131 and 21 in the amino acid sequence shown in SEQ ID NO: 1. “Corresponding to” means corresponding to any of these positions in a related sequence.

As regards the preferred embodiments of an ACH to be mutated according to such a method, the same applies as has been set forth herein-above.

In one preferred embodiment the ACH from which the ACH variant is derived is an ACH which shows the amino acid sequence as shown in SEQ ID NO:1 or an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90% sequence identity to SEQ ID NO:1 or any of the preferred degrees of sequence identity as specified herein above.

Moreover, as regards preferred embodiments of the degree of improvement in activity and the changes to be effected, the same applies as described herein above.

The change(s) which is/are effected at any of the above position(s) is/are substitution(s), deletion(s) and/or insertion(s) as defined herein above.

An ACH variant of the present invention can be fused to a homologous or heterologous polypeptide or protein, an enzyme, a substrate or a tag to form a fusion protein. Fusion proteins in accordance with the present invention will have the same improved activity as the ACH variant of the present invention. Polypeptides, enzymes, substrates or tags that can be added to another protein are known in the art. They may useful for purifying or detecting the proteins of the invention. For instance, tags that can be used for detection and/or purification are e.g. FLAG-tag, His6-tag or a Strep-tag. Alternatively, the protein of the invention can be fused to an enzyme e.g. luciferase, for the detection or localisation of said protein. Other fusion partners include, but are not limited to, bacterial β-galactosidase, trpE, Protein A, β-lactamase, alpha amylase, alcohol dehydrogenase or yeast alpha mating factor. It is also conceivable that the polypeptide, enzyme, substrate or tag is removed from the protein of the invention after e.g. purification. Fusion proteins can typically be made by either recombinant nucleic acid methods or by synthetic polypeptide methods known in art.

The present invention further relates to a nucleic acid molecule encoding an ACH variant of the present invention and to a vector comprising said nucleic acid molecules. Vectors that can be used in accordance with the present invention are known in the art. The vectors can further comprise expression control sequences operably linked to the nucleic acid molecules of the present invention contained in the vectors. These expression control sequences may be suited to ensure transcription and synthesis of a translatable RNA in bacteria or fungi. Expression control sequences can for instance be promoters. Promoters for use in connection with the nucleic acid molecules of the present invention may be homologous or heterologous with regard to its origin and/or with regard to the gene to be expressed. Suitable promoters are for instance promoters which lend themselves to constitutive expression. However, promoters which are only activated at a point in time determined by external influences can also be used. Artificial and/or chemically inducible promoters may be used in this context.

Preferably, the vector of the present invention is an expression vector. Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), lp1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature.

In addition, the present invention relates to a host cell comprising the nucleic acid molecule or the vector of the present invention.

In a preferred embodiment, the host cell according to the presenting invention is a microorganism, in particular a bacterium or a fungus. In a more preferred embodiment, the host cell of the present invention is E. coli, a bacterium of the genus Clostridium or a yeast cell, such as S. cerevisiae. In another preferred embodiment the host cell is a plant cell or a non-human animal cell.

The transformation of the host cell with a vector according to the invention can be carried out by standard methods, as for instance described in Sambrook and Russell (2001), Molecular Cloning: A Laboratory Manual, CSH Press, Cold Spring Harbor, NY, USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc.

The present invention also relates to a method for producing 3-methylcrotonic acid from 3-methylcrotonyl-CoA comprising the step of incubating an ACH variant of the invention with 3-methylcrotonyl-CoA under conditions allowing said conversion or comprising the step of culturing a host cell of the present invention expressing an ACH variant in a suitable medium.

It is also conceivable in this context that in such a method not only one enzyme according to the present invention is employed but a combination of two or more enzymes.

The present invention also relates to the use of an ACH variant or a host cell of the present invention as described above for the conversion of 3-methylcrotonyl-CoA into 3-methylcrotonic acid. Moreover, in a further embodiment, the present invention relates to a method for producing 3-methylcrotonic acid from 3-methylcrotonyl-CoA by bringing 3-methylcrotonyl-CoA into contact with the ACH variant of the present invention or with a host cell comprising a nucleic acid molecule encoding the ACH variant of the present invention. Thus, in a preferred embodiment, the present invention relates to a method for converting 3-methylcrotonyl-CoA into 3-methylcrotonic acid comprising the steps of: (i) culturing the above-described host cell of the invention in a suitable medium; and (ii) achieving the production of 3-methylcrotonic acid from 3-methylcrotonyl-CoA.

The above described methods and uses can also furthermore comprise the steps of enzymatically further converting the produced 3-methylcrotonic acid into isobutene. The thus produced isobutene may be isolated.

Enzymatic means and methods for achieving the conversion of 3-methylcrotonic acid into isobutene are known to the person skilled in the art and are, e.g, described in WO2017/085167.

The present invention also relates to a method for producing crotonic acid from crotonyl-CoA comprising the step of incubating an ACH variant of the invention with crotonyl-CoA under conditions allowing said conversion or comprising the step of culturing a host cell of the present invention expressing an ACH variant in a suitable medium.

It is also conceivable in this context that in such a method not only one enzyme according to the present invention is employed but a combination of two or more enzymes.

The present invention also relates to the use of an ACH variant or a host cell of the present invention as described above for the conversion of crotonyl-CoA into crotonic acid. Moreover, in a further embodiment, the present invention relates to a method for producing crotonic acid from crotonyl-CoA by bringing crotonyl-CoA into contact with the ACH variant of the present invention or with a host cell comprising a nucleic acid molecule encoding the ACH variant of the present invention. Thus, in a preferred embodiment, the present invention relates to a method for converting crotonyl-CoA into crotonic acid comprising the steps of: (i) culturing the above-described host cell of the invention in a suitable medium; and (ii) achieving the production of crotonic acid from crotonyl-CoA.

The thus produced crotonic acid can be enzymatically further converted into other desired compounds or it can be recovered and/or isolated.

In a preferred embodiment, the present invention relates to methods and uses utilizing a host cell of the present invention which expresses an ACH variant of the present invention.

In another preferred embodiment, such a host cell is an organism which is capable of producing 3-methylcrotonyl-CoA and/or crotonyl-CoA.

In another preferred embodiment, the method according to the invention is carried out in culture, in the presence of an organism, preferably a microorganism, producing an enzyme variant of the present invention. In a preferred embodiment, the (micro)organism is recombinant in that the enzyme produced by the host is heterologous relative to the production host. The method can thus be carried out directly in the culture medium, without the need to separate or purify the enzymes. In an especially advantageous manner, a (micro) organism is used having the natural or artificial property of endogenously producing 3-methylcrotonyl-CoA and/or crotonyl-CoA so as to produce 3-methylcrotonic acid and/or crotonic acid directly from the substrate already present in the culture in solution.

In connection with the above described methods and uses, the microorganisms are cultivated under suitable culture conditions allowing the occurrence of the enzymatic reaction of the ACH variants of the present invention. The specific culture conditions depend on the specific microorganism employed but are well known to the person skilled in the art. The culture conditions are generally chosen in such a manner that they allow the expression of the genes encoding the ACH variant of the present invention. Various methods are known to the person skilled in the art in order to improve and fine-tune the expression of certain genes at certain stages of the culture such as induction of gene expression by chemical inducers or by a temperature shift.

In another embodiment, the above described methods of the invention comprise the step of providing the organism, preferably the microorganism carrying the respective enzyme activity or activities in the form of a (cell) culture, preferably in the form of a liquid cell culture, a subsequent step of cultivating the organism, preferably the microorganism in a fermenter (often also referred to a bioreactor) under suitable conditions allowing the expression of the respective enzyme and further comprising the step of effecting an enzymatic conversion of a method of the invention as described herein above. Suitable fermenter or bioreactor devices and fermentation conditions are known to the person skilled in the art. A bioreactor or a fermenter refers to any manufactured or engineered device or system known in the art that supports a biologically active environment. Thus, a bioreactor or a fermenter may be a vessel in which a chemical/biochemical process like the method of the present invention is carried out which involves organisms, preferably microorganisms and/or biochemically active substances, i.e., the enzyme(s) described above derived from such organisms or organisms harboring the above described enzyme(s). In a bioreactor or a fermenter, this process can either be aerobic or anaerobic. These bioreactors are commonly cylindrical and may range in size from litres to hundreds of cubic meters and are often made of stainless steel. In this respect, without being bound by theory, the fermenter or bioreactor may be designed in a way that it is suitable to cultivate the organisms, preferably microorganisms, in, e.g., a batch-culture, feed-batch-culture, perfusion culture or chemostate-culture, all of which are generally known in the art.

The culture medium can be any culture medium suitable for cultivating the respective organism or microorganism.

In yet a further embodiment, the method according to the invention can be carried out in vitro, e.g. in the presence of isolated enzyme or of cell lysates comprising the enzyme or partially purified enzyme preparations comprising the ACH variant of the present invention. In vitro preferably means in a cell-free system.

In one embodiment, the enzyme(s) employed in the method is (are) used in purified form. However, such a method may be costly, since enzyme and substrate production and purification costs are high.

Thus, in another preferred embodiment, the enzymes employed in the method are present in the reaction as a non-purified extract, or else in the form of non-lysed bacteria, so as to economize on protein purification costs. However, the costs associated with such a method may still be quite high due to the costs of producing and purifying the substrates.

In an in vitro reaction the enzymes, native or recombinant, purified or not, are incubated in the presence of the substrate in physicochemical conditions allowing the enzymes to be active, and the incubation is allowed to proceed for a sufficient period of time allowing production of the desired product as described above. At the end of the incubation, one optionally measures the presence of the desired product by using any detection system known to one of skill in the art such as gas chromatography or colorimetric tests for measuring the formation of the product.

In a particularly preferred embodiment of the invention the method is carried out in vitro and the enzyme is immobilized. Means and methods for immobilizing enzymes on different supports are well-known to the person skilled in the art.

The method according to the invention may furthermore comprise the step of collecting gaseous products, i.e. in the case when isobutene is the final product, degassing out of the reaction, i.e. recovering the product which degasses, e.g., out of the culture. Thus, in a preferred embodiment, the method is carried out in the presence of a system for collecting gaseous products, such as isobutene, under gaseous form during the reaction.

As a matter of fact, isobutene adopts the gaseous state at room temperature and atmospheric pressure. Moreover, isobutene also adopts the gaseous state under culture conditions at 37° C. The method according to the invention therefore does not require extraction of isobutene from the liquid culture medium, a step which is always very costly when performed at industrial scale. The evacuation and storage of gaseous hydrocarbons, in particular of isobutene, and their possible subsequent physical separation and chemical conversion can be performed according to any method known to one of skill in the art.

Finally, the present invention relates to a composition comprising a variant of an ACH of the present invention, a nucleic acid molecule of the present invention, a vector of the present invention or a host cell of the present invention. As regards the variant of an ACH, the nucleic acid molecule, the vector or the host cell, the same applies as has been set forth above in connection with the methods according to the present invention.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

FIG. 1: schematically shows the reaction of the enzymatic conversion of 3-methylcrotonyl-CoA into 3-methylcrotonic acid and the reaction of the enzymatic conversion of crotonyl-CoA into crotonic acid

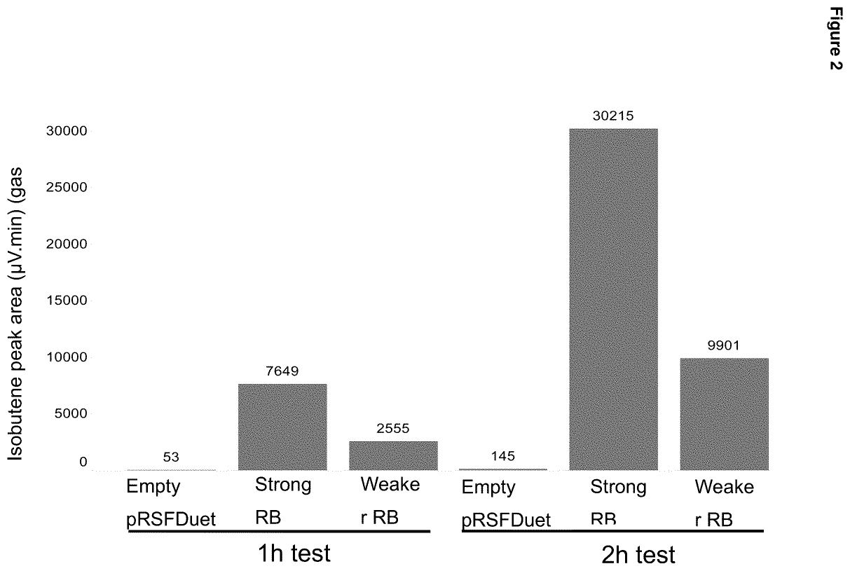

FIG. 2: Optimization of the expression level (Ribosome binding: RB) of the ACH enzyme Ydil of E. coli for the coupled in vivo screening assay.

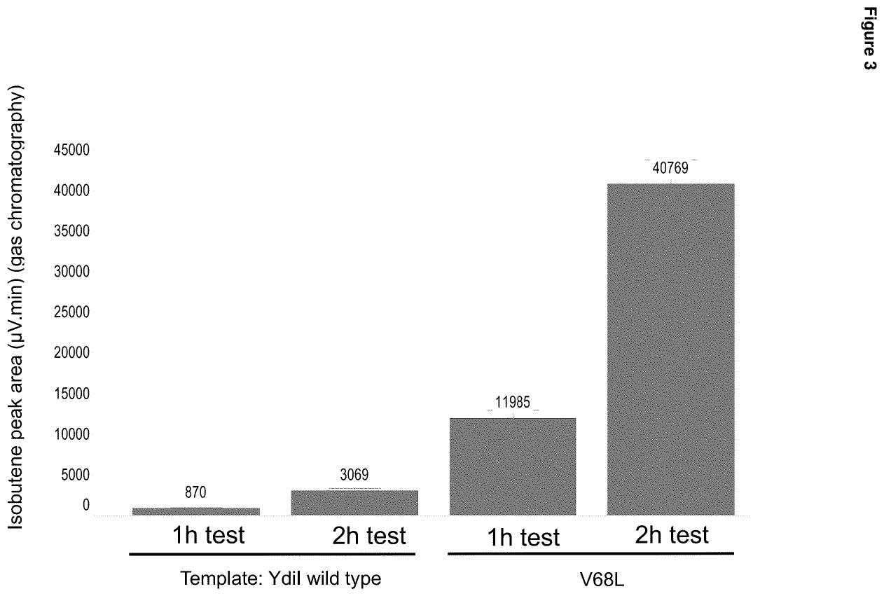

FIG. 3: Beneficial mutation identified after the first round of in vivo screening on the template Ydil wild type enzyme (results of the in vivo coupled assay).

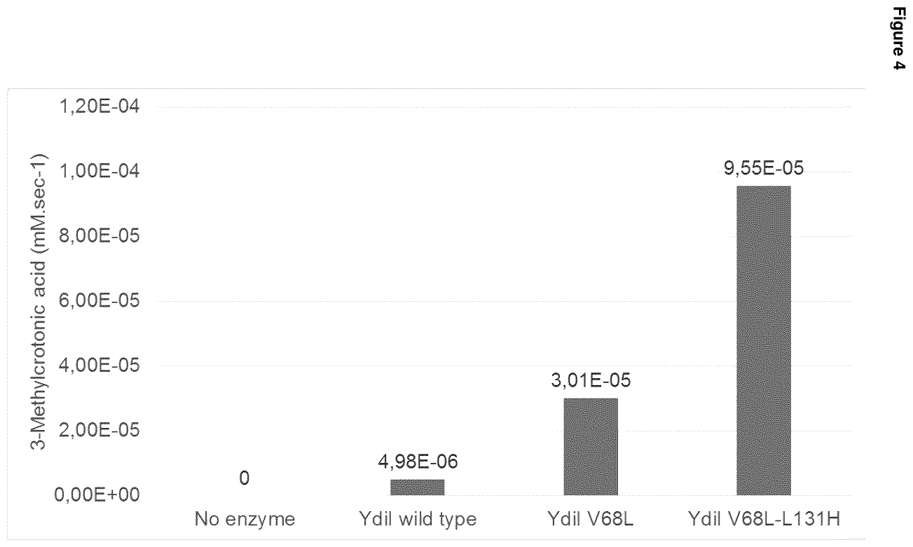

FIG. 4: Comparison between the purified Ydil variants identified after the first and second rounds of screening (results of the in vitro direct assay with 1 mM 3-methylcrotonyl-CoA (MC-CoA)).

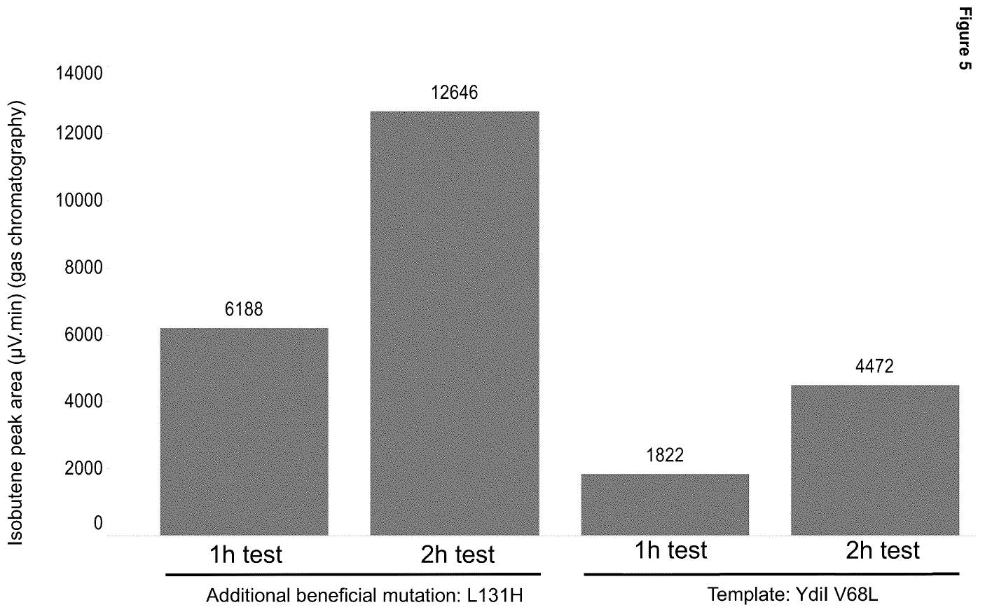

FIG. 5: Additional beneficial mutation identified after the second round of in vivo screening on the template Ydil V68L variant (results of the in vivo coupled assay).

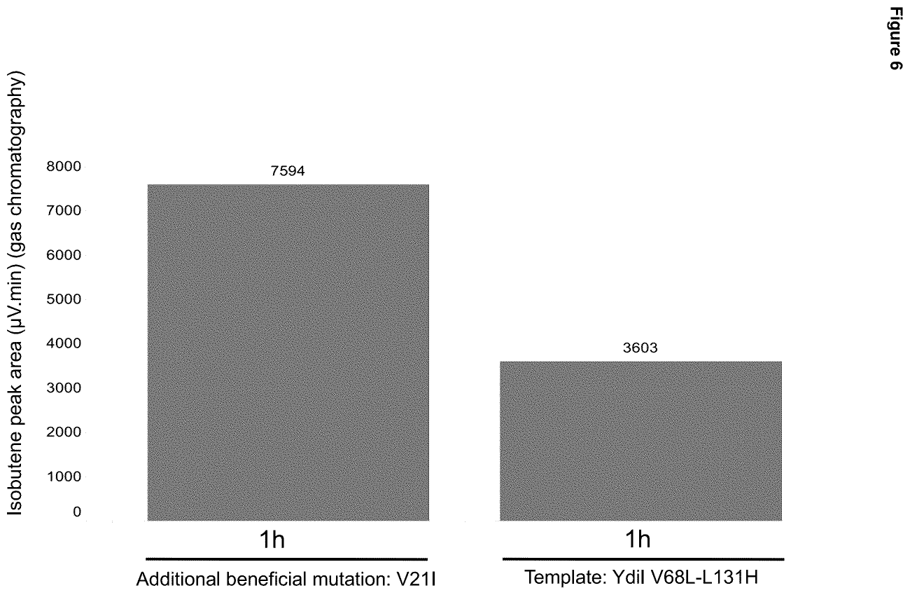

FIG. 6: Additional beneficial mutation identified after the third round of in vivo screening on the template Ydil V68L-L131H variant (results of the in vivo coupled assay).

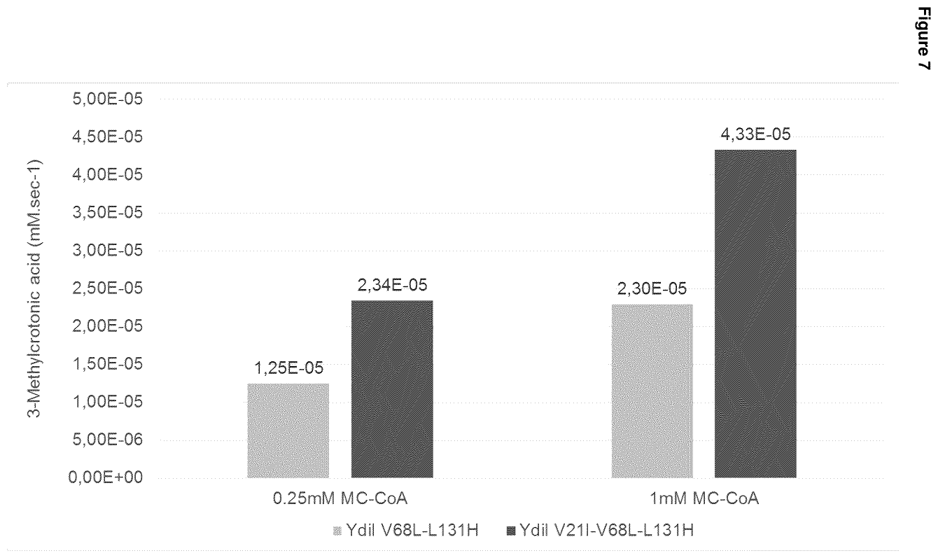

FIG. 7: Comparison between the purified Ydil variants V68L-L131H and V21I-V68L-L131H identified after the third round of screening (results of the in vitro direct assay with 0.25 mM and 1 mM MC-CoA).

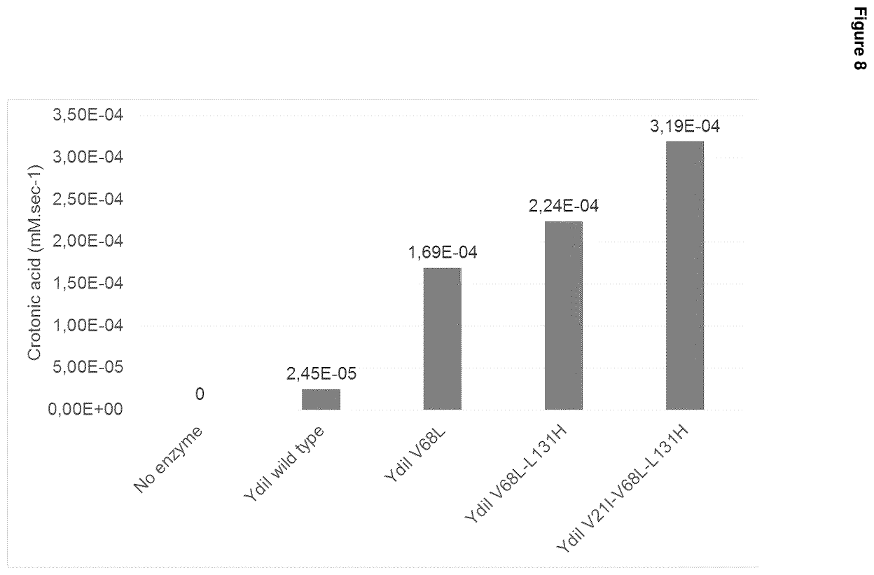

FIG. 8: Comparison between the purified Ydil variants identified after the three rounds of screening (results of the in vitro direct assay with 1 mM crotonyl-CoA).

The invention will now be described by reference to the following examples which are merely illustrative and are not to be construed as a limitation of the scope of the present invention.

### EXAMPLES

### Example 1: Identification of Variants of an Acyl-CoA Hydrolase with Improved Activity of Converting 3-Methylcrotonyl-CoA (MC-CoA) into 3-Methylcrotonic Acid and Verification of Increased Crotonyl-CoA Hydrolase Activity

Ydil (menl) is the wild type 1,4-dihydroxy-2-naphthoyl-CoA hydrolase (EC 3.1.2.28) from Escherichia coli (strain K12) (Uniprot: P77781) catalyzing the hydrolysis of a 1,4-dihydroxy-2-naphthoyl-CoA to 1,4-dihydroxy-2-naphthoate.

This Ydil wild type enzyme was evolved by directed evolution to find improved mutants for the hydrolysis of 3-methylcrotonyl-CoA (MC-CoA) into 3-methylcrotonic acid (FIG. 1). A high MC-CoA hydrolase activity is required to efficiently make 3-methylcrotonic acid or chemical derivatives from the 3-methylcrotonic acid precursor, like isobutene (FIG. 1). Increasing MC-CoA hydrolase activity by directed evolution of a Ydil enzyme also led to Ydil mutants that also have an increased enoyl-CoA hydrolase activity for other substrates, like crotonyl-CoA (see below).

Ydil mutants with improved MC-CoA hydrolase activity were identified by a 2-step approach.

1/Screening of Ydil Mutants with a Coupled in Vivo Assay

This assay is based on the use of a bacterial strain (BL21 (DE3), Novagen) transformed with two compatible expression vectors (pETDuet and pRSFDuet, Novagen) leading to the production of the full recombinant metabolic pathway converting acetyl-CoA to isobutene as described in WO2017085167.

The following enzymes were cloned in the pETDuet expression vector: the HMG-COA synthase from Enterococcus faecalis (Uniprot: Q835L4), the Enoyl-CoA hydratase AtuE from Pseudomonas aeruginosa (Uniprot: Q9HZV7) or from Pseudomonas sp. UW4 (Uniprot: K9NHK2), the MG-CoA decarboxylase AibAB from Myxococcus xanthus (Uniprot: Q1D414 and Q1D413), the 3-Methylcrotonic acid decarboxylase FDC1 from Hypocrea atroviridis (Uniprot: G9NLP8) or from Streptomyces sp. 769(Uniprot: A0A0A8EV26), and the flavin prenyltransferase UbiX from E. coli (Uniprot: POAG03).

The following enzymes were cloned in the pRSFDuet expression vector: the acetyl-CoA acetyltransferase Thl from Clostridium acetobutylicum (Uniprot: P45359) and the evolved MC-CoA hydrolase Ydil from Escherichia coli (strain K12) (Uniprot: P77781). The pRSFDuet vector was designed so that the MC-CoA hydrolase activity was limiting the production of isobutene from glucose. The appropriate expression level of Ydil was identified by decreasing the ribosome binding strength (FIG. 2).

All the amino acid positions (136) of Ydil were mutated by saturation mutagenesis. The 136 mutant libraries were transformed in BL21 (DE3) competent cells containing the pETDuet vector and individual clones were tested for isobutene production from glucose.

The hit clones with improved isobutene production compared to the template were plated from the preculture to confirm the result with several colonies.

2/Purification and Characterization of Ydil Mutants with a Direct in Vitro Assay

The Ydil mutant gene of the clones selected in the screening were sequenced by Sanger sequencing and the selected leads were cloned alone in a pRSFDuet vector to add a 6His purification tag at the N terminus. The Ydil mutants were purified and tested on MC-CoA to produce 3-Methylcrotonic acid in a direct in vitro assay. These purified Ydil mutants were also tested on crotonyl-CoA to produce crotonic acid in a direct in vitro assay, to see if a concomitant increase of Crotonyl-CoA hydrolase activity was also measured.

### RESULTS

The template of the first round of screening was the Ydil wild type enzyme. One beneficial mutation (V68L) was identified after this first round (FIG. 3). This improved mutant Ydil V68L was confirmed as purified protein (FIG. 4) and was used as template for a second round of saturation mutagenesis. One additional beneficial mutation was identified after this second round of saturation mutagenesis: L131H (FIG. 5). This improved mutant Ydil V68L-L131H was confirmed as purified protein (FIG. 4) and was used as template for a third round of saturation mutagenesis. One additional beneficial mutation was identified after this third round of saturation mutagenesis: V21I (FIG. 6). This improved mutant Ydil V21I-V68L-L131H was confirmed as purified protein (FIG. 7). The Ydil mutants with improved MC-CoA hydrolase activity that were identified during the described directed evolution process are listed in Table 1.

TABLE 1
YdiI mutants

| | |
|---|---|
| YdiI V68L | Identified after the 1st round |
| YdiI V68L-L131H | Identified after the 2nd round |
| YdiI V21I-V68L-L131H | Identified after the 3rd round |

These three Ydil mutants also showed an increased Crotonyl-CoA hydrolase activity (FIG. 8).

Additional single point-mutants were created to obtain these three mutations (V21I, V68L and L131H) in all combinations as single, double and triple mutants. Three other mutations with hydrophobic residues were also tested at the first key position that was identified (V68I, V68M, V68F). These new mutants were also tested as purified enzymes with an N-terminal 6His tag and compared to the wild type enzyme for the 3-methylcrotonyl-CoA hydrolase activity at 1 mM (Table 2).

TABLE 2
Improvement factors of YdiI mutants for the activity converting
3-methylcrotonyl-CoA into 3-methylcrotonic acid

| | |
|---|---|
| YdiI wild type | 1 |
| YdiI V21I | 2.95 |
| YdiI V68L | 6.04 |
| YdiI V68I | 5.94 |
| YdiI V68M | 3.92 |
| YdiI V68F | 3.84 |
| YdiI L131H | 4.05 |
| YdiI V21I-V68L | 9.83 |
| YdiI V21I-L131H | 14.34 |
| YdiI V68L-L131H | 19.18 |
| YdiI V21I-V68L-L131H | 36.10 |

### MATERIAL AND METHODS

### Construction of the Ydil Mutant Libraries and Single-Point Mutants

All the enzyme encoding polynucleotide sequences were codon-optimized for the expression in Escherichia coli and subsequently chemically synthesized.

The polynucleotide sequences coding for the different Ydil mutants identified during the directed evolution process were generated using a range of standard molecular biology techniques.

Different PCR-based techniques known in the art were used for the construction of single-point mutants.

Saturation mutagenesis was carried out on the Ydil gene cloned into a pRSFDuet (Novagen) expression vector without fusion to a tag as described above using whole plasmid extension by PCR. The pRSFDuet vector was designed at the ribosome binding site of Ydil so that the MC-CoA hydrolase activity was limiting isobutene production from acetyl-CoA when the full isobutene recombinant pathway was present in the cell. The pRSFDuet expression vector also contained an acetyl-CoA acetyltransferase to produce the acetoacetyl-CoA from two acetyl-CoA molecules (FIG. 1).

### Screening of MC-CoA Hydrolase Activity with a Coupled In Vivo Assay

This is a coupled in vivo assay in 96-well microplates based on isobutene production from glucose.

The strain libraries containing the pRSFDuet and pETDuet expression plasmids were first plated out onto LB-agar plates supplemented with the appropriate antibiotics. Cells were grown overnight at 32° C. until individual colonies reach the desired size. Single colonies were then picked and individually transferred into 50 L of liquid LB medium supplemented with the appropriate antibiotics. Cell growth was carried out with shaking for 20 hours at 32° C. The LB cultures were used to inoculate 1 mL in 96 deep well microplates of auto-induction medium (Studier F W, Prot. Exp. Pur. 41, (2005), 207-234) supplemented with the appropriate antibiotics and grown in a shaking incubator set at 700 rpm and 85% humidity for 24 h at 32° C. in order to produce the recombinant enzymes. The cell pellet containing the full metabolic pathway from glucose to isobutene was then resuspended in 400 μL of minimum medium (pH 7.5, Phosphate 50 mM, Glucose 10 g.L-1, MgSO4 1 mM). The microplate was then thermosealed 5 sec at 180° C. and incubated for a further 1 or 2 hours in a shaking incubator at 36° C., 700 rpm. During this step, the bacterial cell converted glucose into acetyl-CoA with endogenous enzymes, and acetyl-CoA into isobutene with the recombinant enzymes. After 5 min inactivation at 80° C., the isobutene produced was quantified by gas chromatography as followed. 100 μL of headspace gases from each well are injected in a Brucker GC-450 system equipped with a Flame Ionization Detector (FID). Compounds present in samples were separated by chromatography using a RTX-1 column at 100° C. with a 1 mL·min-1 constant flow of nitrogen as carrier gas. Upon injection, peak areas of isobutene were calculated.

### Cloning of Ydil Mutants with an N-Terminal 6His Tag

The polynucleotide sequences coding for the different mutants identified during the evolution of Ydil (in vivo screening) were cloned alone in a pRSFDuet (Novagen) expression vector with a polynucleotide tag in 5′ coding for a 6His purification tag.

### Purification and Characterization of Ydil Mutants with a Direct In Vitro Assay

Ydil mutants were purified by affinity chromatography (Ni-NTA) from bacterial cultures of BL21 (DE3) cells containing the gene cloned into a pRSFDuet (Novagen) expression vector with a polynucleotide tag in 5′ coding for a 6-His purification tag. The purified Ydil mutants were tested at 1 μg/ml or 0.1 μg/ml in a direct in vitro assay with a buffered solution containing 50 mM Tris-HCl pH7.5, 20 mM NaCl, 100 mM KCl, 2 mM MgCl2, 1 mM MC-CoA (Endotherm) (to measure MC-CoA hydrolase activity) or 1 mM crotonyl-CoA (Sigma-Aldrich) (to measure Crotonyl-CoA hydrolase activity). The reactions were incubated 30 min at 36° C. in a water bath, stopped with acetonitrile and the produced 3-methylcrotonic acid (Sigma-Aldrich) (to measure MC-CoA hydrolase activity) or crotonic acid (TCI chemicals) (to measure Crotonyl-CoA hydrolase activity) were quantified by HPLC with a calibration curve. The samples were injected on a Zorbax SB-Aq column (Agilent) at 30° C. and eluted with 8.4 mM H2SO4 and a gradient of acetonitrile.

1. An acyl-CoA hydrolase (ACH) variant comprising a polypeptide at least 60% sequence identity to SEQ ID NO:1, wherein amino acids 68, 131, and/or 121 of SEQ ID NO:1 are substituted, deleted or having an insertion, and wherein said variant has improved activity in:
(a) converting 3-methylycrotonyl-CoA into 3-methylcrotonic acid; or
(b) converting crotonyl-CoA into crotonic acid,
as compared to the ACH polypeptide having the amino acid sequence of SEQ ID NO:1.

2. The ACH variant of claim 1, wherein
(1) the amino acid residue at position 68 in the amino acid sequence shown in SEQ ID NO:1 is deleted or substituted with leucine, isoleucine, methionine or phenylalanine; and/or
(2) the amino acid residue at position 131 in the amino acid sequence shown in SEQ ID NO:1 is deleted or substituted with histidine; and/or
(3) the amino acid residue at position 21 in the amino acid sequence shown in SEQ ID NO:1 is deleted or substituted with isoleucine.

3. A nucleic acid molecule encoding the ACH variant of claim 1.

4. The nucleic acid molecule of clim 3, wherein the nucleic acid molecule further comprises vector sequence.

5. The nucleic aci molecule of claim 3, wherein the nucleic acid molecule is contained with a host cell.

6. A method for producing 3-methylcrotonic acid from 3-methylcrotonyl-CoA comprising incubating 3-methylcrotonyl-CoA with the ACH variant of claim 1 to produce 3-methylcrotonic acid.

7. A method for producing crotonic acid from crotonyl-CoA comprising incubating the ACH variant of claim 1 with crotonyl-CoA to produce crotonic acid.

8. A method for producing 3-methylcrotonic acid from 3-methylcrotonyl-CoA comprising incubating 3-methylcrotonyl-CoA with the ACH variant of claim 2 to produce 3-methylcrotonic acid.

9. A method for producing crotonic acid from crotonyl-CoA comprising incubating crotonyl-CoA with the ACH variant of claim 2 to produce crotonic acid.

10. The method of claim 6, wherein the enzymatic conversion is carried out in vitro.

11. A composition comprising a variant of an ACH of claim 1.

12. The composition of claim 11, wherein the composition further comprises methylcrotonyl-CoA and/or crotonyl-CoA.

13. The nucleic acid molecule of claim 4, wherein the nucleic acid molecule and the vector sequence is contained with a host cell.

14. The method of claim 7, wherein the enzymatic conversion is carried out in vitro.

15. The method of claim 8, wherein the enzymatic conversion is carried out in vitro.

16. The method of claim 9, wherein the enzymatic conversion is carried out in vitro.

17. The composition of claim 12, wherein the composition comprises 3-methylcrotonyl-CoA.

18. The composition of claim 12, wherein the composition comprises crotonyl-CoA.
